(19) United States
(12) Patent Application Publication
(10) Pub. No.: US 2007/0219492 A1
(43) Pub. Date: Sep. 20, 2007

(54) Injection Syringe

(76) Inventors: Jan Hendrik Lucas, Brecht/St. Job (BE); Bjorn Olaf Goossens, Den Haaag (NL)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/592,960

(22) PCT Filed: Mar. 8, 2005
(86) PCT No.: PCT/NL05/00168

§ 371 Date: Jan. 2, 2007

(30) Foreign Application Priority Data
Mar. 9, 2004 (NL) 1025669

(51) Int. Cl. A61M 5/50

(52) U.S. Cl. 604/110

(57) ABSTRACT

An injection syringe with retractable injection needle has a liquid container with a needle opening, as well as a plunger/plunger rod assembly which is moveable over a travel in the liquid container. Furthermore, an injection needle is provided, which in an active position projects out of the liquid container through the needle opening and in a retracted position is located fully within the liquid container. The injection syringe has a travel limiter which is activated after an injection has been administered using the injection syringe and subsequently the injection needle with needle mount has been moved into the retracted position by the plunger/plunger rod assembly being retracted, after which it restricts the plunger/plunger rod assembly in the event of a movement towards the needle opening, in such a manner that the injection needle cannot be forced out of the liquid container.

The present invention relates to an injection syringe having a retractable injection needle. An injection syringe of this type usually comprises a liquid container with a needle opening, a plunger/plunger rod assembly which is moveable within the liquid container, and an injection needle with associated needle mount.

In an active position, the injection needle projects out of the liquid container through the needle opening, and the needle mount is locked to the liquid container.

In an embodiment which is generally known, coupling is effected between the plunger head and the needle mount when the plunger/plunger rod assembly has moved ail the way inwards in order to discharge all the liquid from the injection syringe. As a result of the returning of the plunger rod, the injection needle is then pulled into the inside of the liquid container until the injection needle is located completely within the liquid container in a retracted position. In this retracted position, the risk of injury on the needle or contamination with a transmissible disease by the injection needle is reduced.

One problem of known embodiments of this type of injection syringe is that after the injection needle has been retracted into the liquid container, the plunger can intentionally or unintentionally be moved back towards the needle opening, which means that there is some risk of the injection needle puncturing the (generally plastic) wall of the liquid container. This negates the protective effect of retracting the injection needle into the liquid container, which means that it is possible for a user to injure himself on the injection needle or to catch a transmissible disease through contact. It will be clear that this is highly undesirable.

It is evident from the prior art that it has been attempted to solve this problem by making the wall of the liquid container thicker or stronger However, it has emerged that it is in this way still not always possible to prevent the injection needle—after it has been retracted into the liquid container—from still puncturing the wall of the liquid container.

Another way in which it has been attempted to solve this problem is by using what is known as a travel limiter when designing the injection syringe. In a known injection syringe, a travel limiter based on a friction mechanism is provided; this travel limiter is activated after an injection has been administered using the injection syringe and subsequently the injection needle with needle mount has been moved into the retracted position by the plunger/plunger rod assembly being retracted, after which it prevents any movement of the plunger/plunger rod assembly towards the needle opening, in such a manner that the injection needle cannot be pressed out of the liquid container. This known injection syringe with travel limiter has also not been found to function satisfactorily.

It is an object of the present invention to provide an improved injection syringe which at least partially eliminates the above drawbacks, and/or to create a useable alternative.

Another object of the invention is to provide an injection syringe (in particular with a liquid volume of less than 5 ml, preferably less than 2 ml, even more preferably less than 1 ml, such as 0.5 ml) with retractable injection needle which is simple and inexpensive to produce and is also simple and safe to use.

The invention provides an injection syringe with retractable injection needle in accordance with the preamble of claim L, the travel limiter comprising a stop mechanism having a stop face associated with the plunger/plunger rod assembly, and having a stop face associated with the liquid container. In this case, at least one of these stop faces is moveable between an initial, inactive position, in which an injection can be administered using the injection syringe, and an active position, in which the stop faces come into contact with one another and limit the travel of the plunger/plunger rod assembly with respect to the liquid container.

The stop mechanism prevents further movement of the plunger/plunger rod assembly beyond the stop point defined by the stop mechanism in a safe and reliable way, thereby preventing the possibility of the injection needle puncturing the wall of the liquid container.

Given a suitable design of the stop mechanism (including dimensions and quality of the components used), it is possible to achieve a considerably improved reliability compared to a limiter based on friction. For example, when using friction the friction blocking can be overcome by the application of a large force. Although such situations will generally involve improper use, the potential consequences are such that this has to be regarded as a serious danger in the field of injection syringes.

Another factor linked to the above is that—in the position in which the stop faces of the stop mechanism bear against one another—the part of the plunger rod which projects out of the liquid container does not have to be unnecessarily long in the case of the injection syringe according to the invention. After all, the stop mechanism merely has to guarantee that a “protected space” of sufficient length remains free in the liquid container for the unit made up of needle and needle mount. Should the plunger rod be pulled out further than this by the user, the user can push it back inwards until the stop faces come into contact with one another. In this situation, the injection syringe has an acceptable, relatively short length and can easily be disposed of, for example in a bin or the like for used injection syringes. This avoids a problem which arises with the known injection syringe with the friction-based limiter, whereby once a plunger rod which has been pulled a long way out of the liquid container, it is immediately blocked such that it cannot be pushed inwards again, and consequently a very long injection syringe then has to be disposed of. This is difficult, and also users then still tend to shorten the length, for example by exerting an excess force on the plunger rod as explained above.

Another advantage of the travel limiter designed as a stop mechanism is that the plunger rod—before the injection needle is retracted into the liquid container—can be moved back and forth a number of times, which may be necessary, for example, in order to homogenize liquid that is present in the liquid container or to mix two or more liquids in the liquid container.

Another advantage is that the stop mechanism only becomes active after the injection liquid has been discharged, so that all the liquid can be reliably discharged from the liquid container, which is highly desirable in particular in the case of expensive liquids.

The injection syringe according to the present invention can be of relatively simple design and can be produced easily and at low cost using a small number of components.

The injection syringe with retractable injection needle can be designed in various ways in order to enable the injection needle to be retracted into the liquid container after the liquid container has been emptied. For this purpose, the needle mount and the plunger head may, for example (and preferably), be provided with coupling means. The injection syringe can also, in combination with or as an alternative to the coupling means, be provided with spring means which can push or pull the injection needle into the liquid container.

The injection syringe may be a prefilled injection syringe.

Furthermore, the injection syringe can be provided with seals at various points in order to ensure that the injection syringe is free of leaks and that no contamination of liquid will appear in the liquid container.

To hold the injection needle in the active position, it is known to provide locking means, which preferably lock the needle mount with respect to the liquid container. Numerous embodiments of locking means of this type are known. The locking means can also be unlocked, in such a maimer that the injection needle with needle mount can move into their retracted position in the liquid container.

When the injection syringe according to the present invention is in use, the injection syringe is first of all used to inject liquid that is present in the liquid container into a patient or the like. For the sake of completeness, it should be noted here that the injection syringe, if desired, prior to injection may be used to suck up liquid out of a vial or the like. After the liquid container has been emptied, the plunger/plunger rod assembly is moved back, and as a result of the coupling to the needle mount obtained, or in some other way (for example under the influence of spring means), the injection needle with needle mount is moved to the retracted position in the liquid container. In the process, the locking means associated with the injection needle are unlocked.

The stop mechanism is activated at some time during this movements of the plunger/plunger rod assembly.

If the plunger rod is moved inwards again after the injection needle has been retracted into the liquid container, the plunger rod (on account of the fact that the stop mechanism is activated) will be blocked at one or more predetermined stop positions, and the injection needle can no longer puncture the wall of the liquid container.

As has already been stated above, according to the invention it is preferable for the injection syringe to comprise coupling means for coupling the plunger head to the needle mount after, or actually during, the emptying of the liquid container as a result of the plunger being moved inwards. These coupling means can be designed in numerous different ways. By way of example, the plunger head may be provided with a recess which can interact with one or more small claws or other coupling projections which are secured to the needle mount. In this case, the plunger lead can also be used to unlock the needle mount with respect to the liquid container. In an alternative embodiment, the needle mount may itself be provided with a recess which can interact with one or more coupling projections on the plunger head.

The injection syringe is preferably provided with a removable protective cap which, prior to use, protects that part of the plunger/plunger rod assembly which projects out of the liquid container. This protective cap prevents the possibility of the plunger rod being moved inadvertently. If a protective cap is also fitted over the injection needle, it may be possible to do without any further packaging for the injection syringe.

In a further aspect, the present invention relates to a plunger rod, a travel limiter and a liquid container, described as components of the injection syringe according to the invention.

In yet another aspect, the present invention relates to a set made up of a liquid container, a plunger rod and a travel limiter described as components of the injection syringe in accordance with the invention. In principle, this set can also be used for injection syringes which do not employ a retractable injection needle but for which it is desirable for it to be possible to block the plunger rod.

The present invention will be explained below on the basis of the appended, non-limiting drawing, in which:

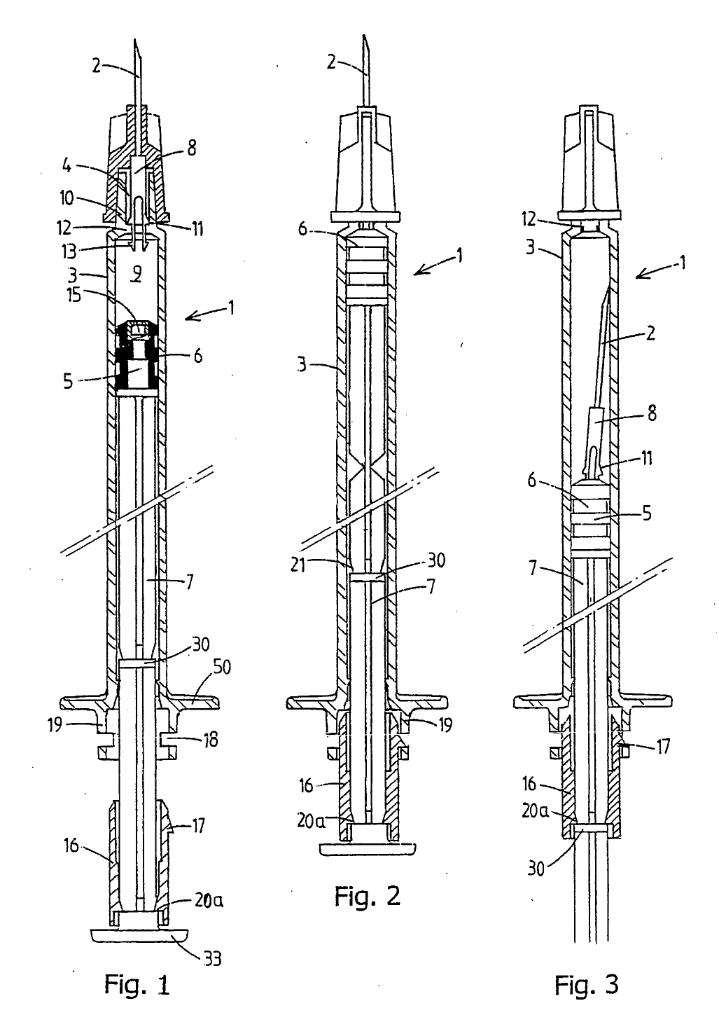

FIG. 1 shows a diagrammatic cross section through an injection syringe in accordance with the present invention, with the liquid which was originally present in the liquid container having been partially discharged;

FIG. 2 shows the injection syringe in accordance with FIG. 1, with the plunger head having been coupled to the needle mount and the travel limiter having been activated;

FIG. 3 shows the injection syringe in accordance with FIGS. 1 and 2, with the injection needle with needle mount having been received in the liquid container; and

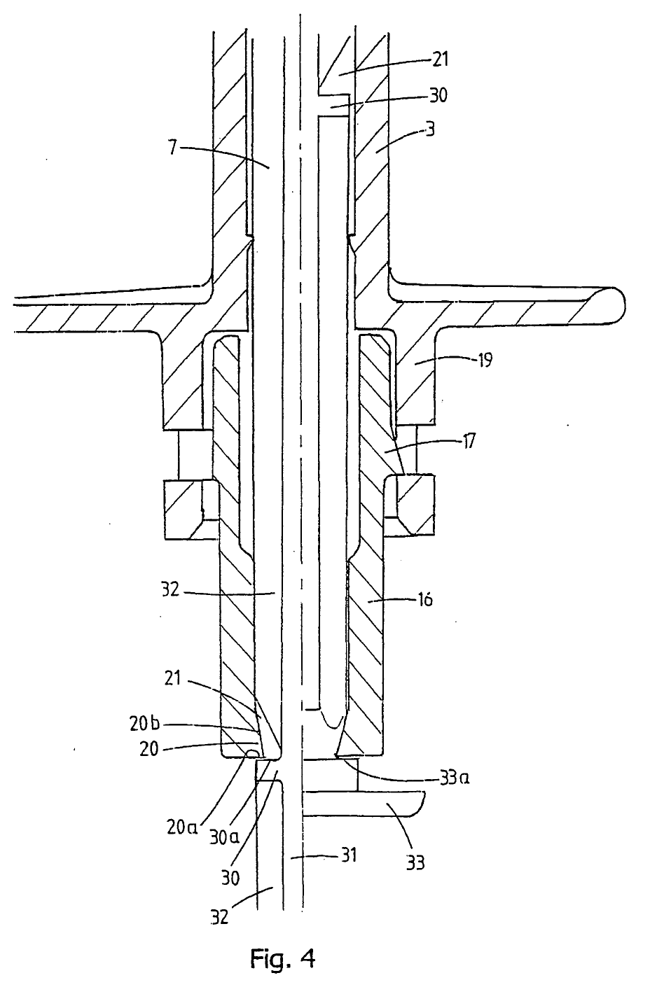

FIG. 4 shows an enlarged partial view of the travel limiter in accordance with FIGS. 1-3;

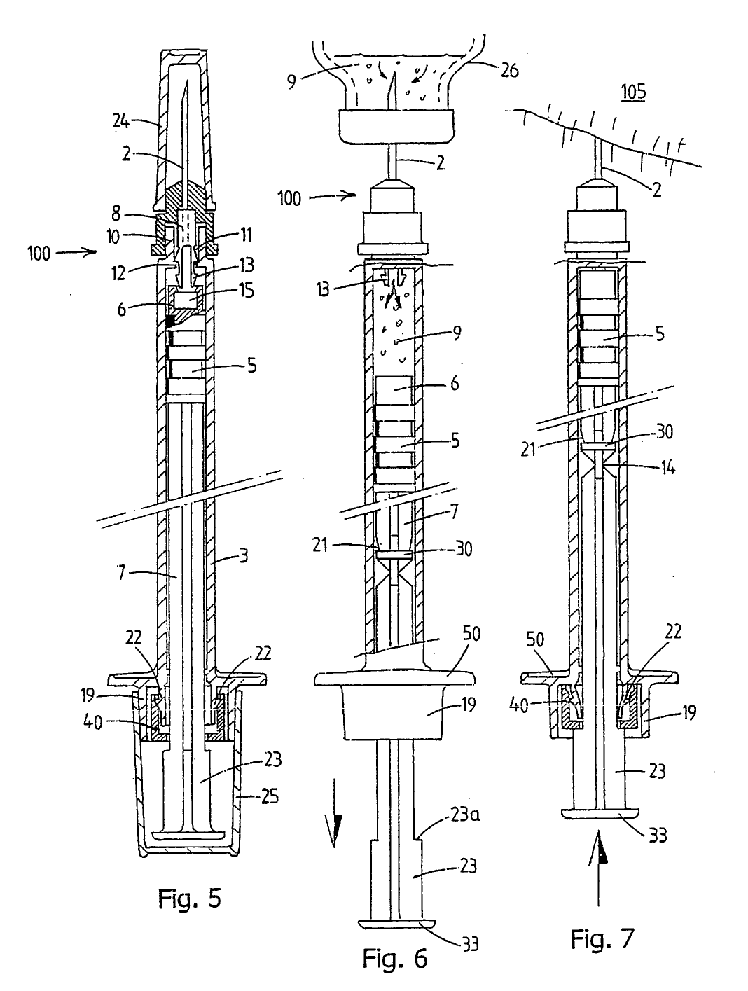

FIG. 5 shows a diagrammatic cross section through an alternative injection syringe in accordance with the invention, in its position prior to use;

FIG. 6 shows the injection syringe in accordance with FIG. 5 while it is sucking up a liquid;

FIG. 7 shows the injection syringe in accordance with FIGS. 5 and 6 during injection;

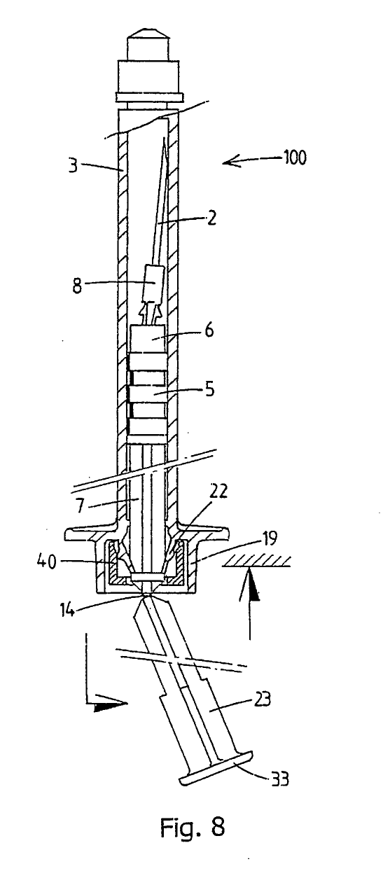

FIG. 8 shows the injection syringe in accordance with FIGS. 5-7 with the injection needle together with needle mount having been received in the liquid container;

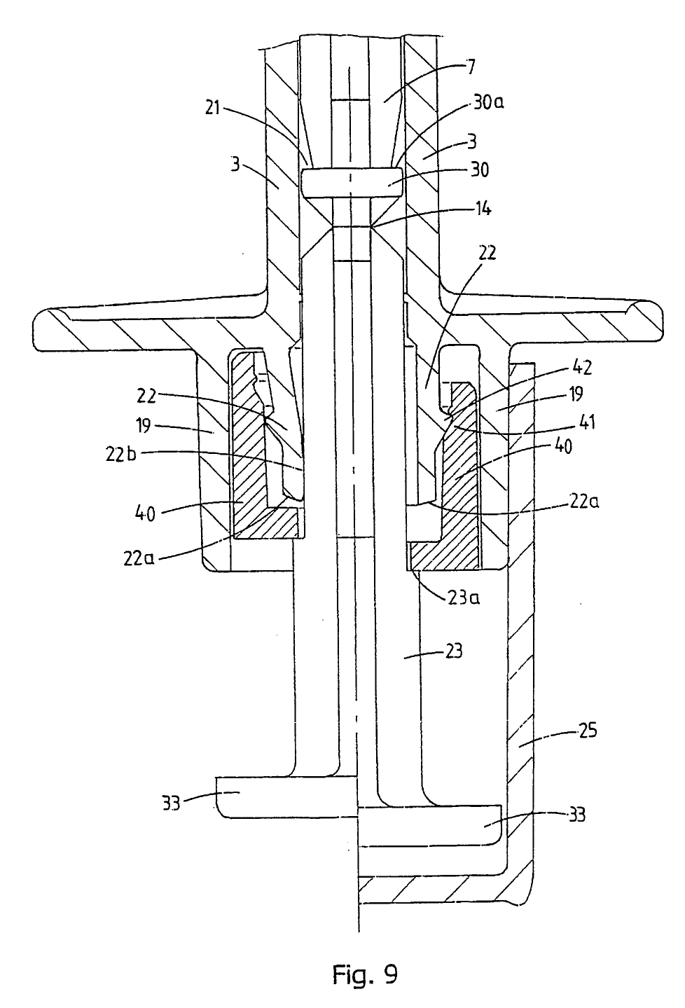

FIG. 9 shows an enlarged partial view of the travel limiter in accordance with FIGS. 5-8;

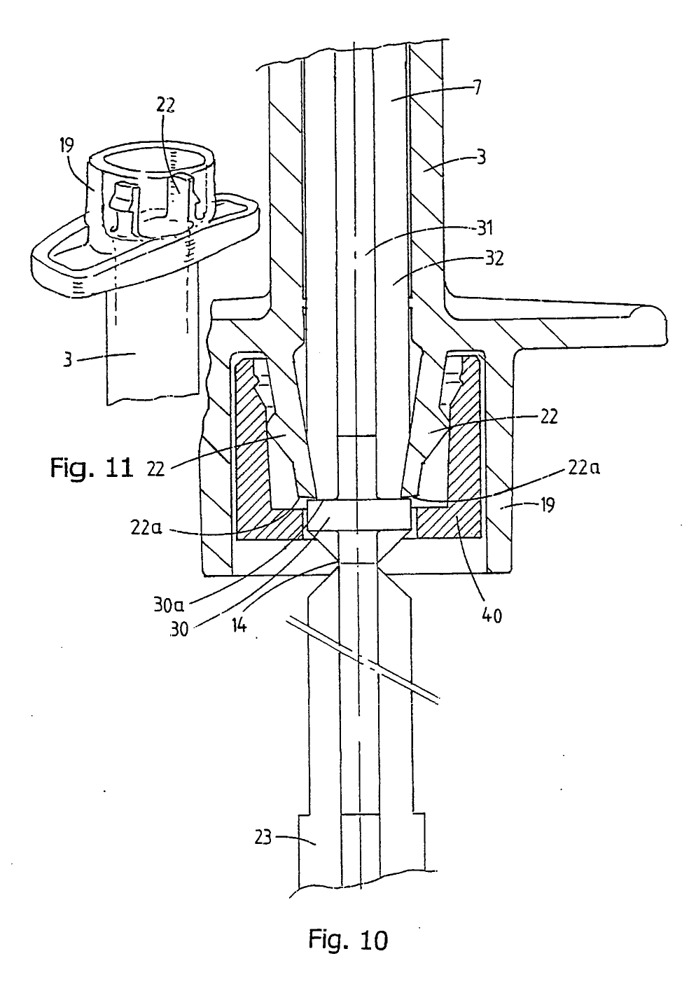

FIG. 10 shows an enlarged partial view of the travel limiter in accordance with FIGS. 5-8, with the recess 21 having moved past the blocking fingers 22;

FIG. 11 shows a schematic partial view of the liquid container 3 in accordance with FIGS. 5-8; and

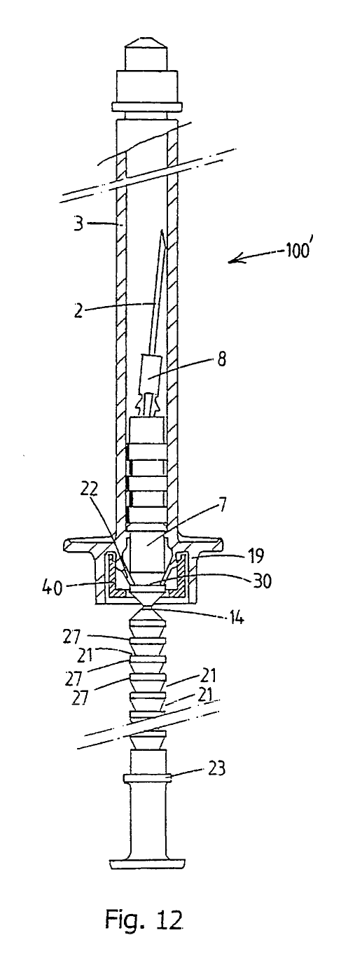

FIG. 12 shows a diagrammatic cross section through a further alternative injection syringe in accordance with the invention, with the plunger rod 7 provided with a series of recesses 21.

Throughout the figs., identical reference numerals denote similar components.

A first exemplary embodiment of an injection syringe according to the invention will be explained with reference to FIGS. 1-4. FIGS. 1-3 show a diagrammatic cross section through an injection syringe 1 with retractable injection needle 2.

The injection syringe 1 has a liquid container 3, which in this case has a substantially tubular body, with a needle opening 4, which can also be described as the outlet opening, at one end of the said tubular body. The tubular body is open at the other end, where a plunger/plunger rod assembly fits into the liquid container. On this side there is also a finger support 50, so that a finger can be placed on either side of the tubular body for supporting purposes. This support 50 can be designed as a (circular) plate body projecting radially outwards.

The plunger/plunger rod assembly is moveable to and fro in the liquid container 3 and has a plunger 5 which fits in a sealing manner in the tubular body, and a plunger rod 7, which is secured to the plunger 5. Furthermore, the assembly has a plunger head 6 on the side facing the needle opening 4.

In FIGS. 1 and 2, the injection needle 2 projects outwards in its active position, with the associated needle mount 8 located at the needle opening 4 in the liquid container 3.

The injection syringe 1 has a retractable injection needle 2, i.e. the injection syringe 1 is designed in such a manner that the injection needle 2 with needle mount 8, after liquid 9 that was present in the liquid container 3 has been substantially emptied out, can be retracted into the liquid container 3 into a retracted position, in which the needle with needle mount is completely inside the liquid container (cf. FIG. 3).

For the injection needle 2 to be retracted, the plunger head 6 and the needle mount 8 are provided with interacting coupling means (in FIG. 1: small claws 13 on the needle mount 8 and the recess 15 in the plunger head 6), which can be designed in various ways, as is also evident from the relevant patent literature.

As a result of the injection needle 2 being retracted into the liquid container 3 after the injection has been administered, the user can no longer injure himself on the injection needle 2.

The needle opening 4 in the liquid container 3 comprises a narrowed section 10 (generally a standard cone). The narrowed section 10 forms part of the liquid container 3 and is fixedly connected thereto, thereby contributing to improved sealing of the injection syringe 1.

In this example, the needle mount 8 with injection needle 2 is locked in its active position in the needle opening 4 of the liquid container 3, in this case by means of projecting locking members 11 which interact with corresponding protuberances 12 in the narrowed section 10. In the embodiment shown, the locking members 11 can be unlocked by coupling the (recess 15 of the) plunger head 6 to the claws 13 of the needle mount 8, facing towards the plunger head 6 of the injection syringe 1 and then moving the plunger rod 7 away from the needle opening 4.

The presence of the locking means 11 means that a relatively high force can be exerted on the injection needle 2 when administering an injection to a patient without any risk of the needle mount 8 unintentionally being pushed out of the needle opening 4 into the liquid container 3. When a force of this nature is applied during injection, the locking members 11, given a suitable design, will in fact become more secure.

The injection syringe 1 has a travel Limiter which is activated after an injection has been administered using the injection syringe and subsequently the injection needle together with needle mount has been moved into the retracted position by retracting the plunger/plunger rod assembly, after which it restricts the plunger/plunger rod assembly in the event of a movement towards the needle opening, in such a manner that the injection needle cannot be forced out of the liquid container. The way in which this travel limiter works will already be clear to the person skilled in the art from FIGS. 1-4 and will be explained in detail below.

The travel limiter is based on a stop mechanism having a stop face 30a associated with the plunger/plunger rod assembly, and having a stop face 20a associated with the liquid container.

The stop face 20a is moveable between an initial, inactive position (FIG. 1), in which an injection can be administered using the injection syringe, and an active position (FIGS. 2, 3), in which the stop faces 20a, 30a come to bear against one another and restrict the travel of the plunger/plunger rod assembly.

The stop face 20a associated with the liquid container is in this case formed by stop element 16, which is guided displaceably on the plunger rod 7, in this case that part of the plunger rod which projects out of the liquid contained, the stop element 16 in this example being an annular stop element 16 which is located around the plunger rod and can also be designed as a cylindrical member which is positioned around the plunger rod.

The stop element 16 and the liquid container 3, on the side remote from the needle opening 4, are provided with interacting coupling means, which are designed in such a manner that during the inward movement of the plunger/plunger rod assembly for discharging liquid from the injection syringe, the stop element 16 is coupled to the liquid container 3.

In the embodiment shown, the stop element 16 is provided with at least one outwardly directed claw 17 which can engage in a designated opening 18 in a widened portion 19 of that end of the liquid container 3 which is remote from the needle opening 4, as can be seen from FIG. 4.

At the end remote from the needle opening, the stop element 16 is provided with one or more inwardly directed stops 20, also referred to as plunger rod blockers or projections, which each form a stop face 20a and can interact with a recess 21 on the plunger rod 7.

The plunger rod 7 is provided with recess 21 for each of the projections 20.

In the embodiment shown, the plunger rod 7 is cross-shaped, with longitudinal ribs 31 and longitudinal grooves 32, over part of its length, as is known per se. The recesses 21 are arranged in each case of the ribs 31. The recesses 21 open towards the side, so that a projection 20 can enter a recess 21. One side of the recesses 21 in each case forms a stop face 30a for the projection 20, and these stop faces 30a are in this case formed by a circular disc formation 30, which is integral with the plunger rod and extends into the longitudinal grooves 32.

As can be seen from FIGS. 2 and 3, as the plunger/plunger rod assembly is being retracted, the recesses 21 move past the projections 20 of the stop elements 16, in such a manner that when the plunger/plunger rod assembly is moved inwards again, the stop faces 20a, 30a come to bear against one another (cf. FIGS. 3 and 4). In this example, the projections 20 can move elastically between an outer position (in which a longitudinal rib 32 of the plunger rod slides along a projection) and an inner position (in which they engage in a recess 21). It can be seen that the projections 20 are provided with inclined surfaces 20b on their side facing towards the plunger rod.

It can also be seen in FIG. 1 that the plunger rod 7 is also provided with recesses (substantially corresponding to the recesses 21) in the vicinity of push-button 33, with which recesses the projections 20 are in engagement in order to releasably lock the stop element 16 with respect to the plunger rod in the initial position of the injection syringe, namely in a position against the push-button 33. If appropriate, other locking means may also be provided for holding the stop element 16 in the initial position in the vicinity of the push-button 33.

When the injection syringe 1 shown in FIGS. 1-4 is in use, the injection syringe 1 will be emptied by the liquid 9 present in the liquid container 3 being forced through the injection needle 2 as a result of the plunger 5 being moved towards the opening 4. If desired, before being emptied, the injection syringe 1 can first be used to suck up liquid out of a vial. (cf. FIG. 6). When the injection syringe 1 is substantially empty, i.e. when the plunger head 6 has been pressed as far as possible inwards, the plunger head 6 of the plunger 5 will be coupled to that side of the needle mount 8 which faces the plunger head 6. The plunger head 6 and the needle mount 8 will interact with one another so as to effect coupling. This state is shown in FIG. 2.

As can be seen clearly from FIG. 2, the widened section 19, designed as an annular wall with a larger diameter than that of the tube part of the liquid container 3, at least partially receives the stop element 16 as a result of the stop element here partially projecting into the annular wall.

Furthermore, the stop element 16 is in the process fixedly connected to the liquid container 3 by virtue of the interaction between the claws 17 of the stop element 16, on the one hand, and the opening(s) 18 in the widened section 19 of the liquid container 3, on the other hand This activates the stop element 16.

When the plunger 5 is then pulled away from the opening 4 again with the aid of the plunger rod 7, the locking members 11, also referred to as blocking members 11, are unlocked or unblocked, and the needle mount 8 together with injection needle 2 can be pulled into the liquid container 3.

To enable the stop element 16 to execute its blocking action, the plunger rod 7 has to be retracted sufficiently fax for the recesses 21 in the plunger rod 7 to move past the projections 20 of the travel limiter 21 (cf. FIG. 3).

If the plunger rod 7 is then moved back towards the opening 4 (with the recess 21 located beyond the plunger rod blockers 20—cf. FIG. 3 once again), the plunger rod 7 will be blocked. As a result, therefore, the effective length of the plunger rod 7 is shortened and the injection needle 2 can no longer easily puncture the wall of the liquid container 3. This therefore minimizes the risk of injury on the injection needle 2 and the risk of transmission of contagious diseases.

The person skilled in the art will quickly understand that the position of the recesses 21 on the plunger rod 7 determines how far the plunger rod 7 has to be retracted to activate the stop mechanism. This position of the recesses 21 can be selected as desired. The position of the recesses 21 is at least selected in such a way that when the recesses 21 have moved past the plunger rod blockers 20 and are located beyond the projections 20 (with respect to the liquid container), the injection needle 2 with needle mount 8 is located within the liquid container 3.

FIG. 4 shows an enlarged partial view of the injection syringe shown in FIGS. 1-3. The right-hand side of FIG. 4 shows the plunger rod in the position indicated in FIG. 2, in which the coupling of the stop element 16 to the liquid container 3 has been realized on account of the fact that an actuating surface 33a, in this case formed by a push-button 33 on the end of the plunger rod, has pressed the stop element 16 towards the liquid container and produced the coupling.

The left-hand side of FIG. 4, on the other hand, shows the situation in FIG. 3, in which the recesses 21 have moved past the projections 20 and the stop faces 20a, 30a are blocking the plunger rod 7 from being pressed further inwards.

It will be clear that on account of the projections 20 and the recesses 21 being shaped differently, it is also possible to achieve the effect whereby if the projections 20 latch into the recesses, the plunger rod is blocked in both directions and therefore can also no longer be pulled outwards.

The person skilled in the art will quickly understand that the injection syringe shown in FIGS. 1-4 can be varied in numerous ways without departing from the scope of the invention. For example, the plunger rod 7 may be provided with a break-off zone, for example in order to enable that part of the plunger rod which projects out of the stop element 16 to be broken off.

The injection syringe 1 may also be provided with a spring member which can help with moving the injection needle 2 with needle mount 8 into the liquid container 3 after the needle mount 8 has been uncoupled from the opening 4 of the liquid container 3.

If appropriate, the plunger rod 7 may be provided with a series of recesses 21 distributed over the length of the plunger rod 7.

An alternative injection syringe 100 according to the invention will now be explained with reference to FIGS. 5-11. Corresponding or similar components to those of the injection syringe 1 are provided with the same reference numeral.

The injection syringe 100 is likewise provided with a travel limiter which is designed as a stop mechanism, is activated after an injection has been administered using the injection syringe and subsequently the injection needle with needle mount has been moved into the retracted position by the plunger/plunger rod assembly being retracted, after which it restricts the plunger/plunger rod assembly (5) in the event of a movement towards the needle opening (4), in such a manner that the injection needle cannot be pressed out of the liquid container.

In this embodiment, the liquid container 3 is provided, at its end remote from the needle opening 4, with two fingers 22, which are in this case integral with the (plastic) body of the liquid container. The fingers 22 form the moveable stops, associated with the liquid container 3, of the stop mechanism that is to be explained, the fingers 22 each forming a stop face 22a. The fingers 22 are resilient and can therefore be moved between an inactive position, located towards the outside with respect to the plunger rod (cf. FIG. 5) and an active position located towards the inside (cf. FIG. 7). The fingers 22 have the same function of blocking the plunger rod as the plunger rod blockers or projections 20 of the injection syringe 1 shown in FIGS. 1-4.

As can be seen in FIG. 1, the fingers 22 are arranged around the opening for the plunger/plunger rod assembly on that side of the liquid container 3 which is remote from the needle opening 4.

An annular activation element 40 is located around the fingers 22, in this case within an annular wall 19 at that end of the liquid container 3 which is remote from the needle opening 4.

The annular wall 19 in this case has a larger internal diameter than the tubular part of the liquid container. The annular wall 19 in this case in part serves to hold the protective cap 25, which is pushed over and clamped onto the annular wall 19, in place.

In the embodiment shown, the activation element 40 is accommodated in the space between the annular wall 19 and the plunger rod 7.

It can be seen that the activation element 40 in this case does not project beyond the annular wall 19 (even in the initial position shown in FIG. 5), so that the activation element 40 is actually “hided ” from the user. Given a suitable design, the user cannot use his finger to operate the activation element, and the tendency to do this is also counteracted by the “hided” arrangement.

The activation element 40 is in this case axially displaceable with respect to the liquid container 3. This displacement is in this case realized by a thickened portion 23 in the vicinity of the push-button 33, which thickened portion, on its side facing towards the liquid container 3, forms an actuating surface 23a for the activation element 40.

In this example, the activation element 40 is provided with a positioning member, in this case a groove 41 on the inner circumference, which holds the activation element 40 in a defined position with respect to the stops 22 prior to the displacement of the activation element 40. In the embodiment shown, outwardly directed projections 42 on the fingers 22 engage in this groove 41 (cf. the right-hand side of FIG. 9). It will be clear that in this defined position the activation element 40 does not level with or towards the inside with respect to the free end of the annular wall 19.

As the plunger rod 7 moves inwards, the actuating surface 23a comes into contact with the activation element 40, so that the activation element 40 is carried along with the plunger rod 7.

On account of the fact that the projections 42 are now no longer located in the groove 41, the projections 42 run along a part with a smaller internal diameter of the activation element 40, so that the fingers 22 bend inwards (cf. the left-hand side of FIG. 9). In this case, the inwardly facing surface 22b of the fingers 22 bears against the ribs 31 of the plunger rod 7.

The activation element 40 reaches its limit position (left-hand side in FIG. 9) when the plunger/plunger rod assembly has been displaced all the way inwards for the purpose of delivering an injection.

This “actuation” of the activation element as a result of the plunger/plunger rod assembly being pressed inwards preferably coincides with the production of the coupling between the plunger/plunger rod assembly and the needle mount. It is preferable for both actions to take place during the last few millimetres of the travel of the plunger rod. This means that if the user does not completely empty the injection syringe, no coupling is effected but also the stop mechanism does not come into effect. The user can then discharge the remaining liquid somewhere in a suitable way by completely emptying the injection syringe, and then the intended effect is achieved.

Preferably, however, the displacement of the activation element only requires a small amount of force, so that the user clearly continues the emptying movement all the way to the end. In the embodiment shown, this can be realized by virtue of the fact that the fingers can be relatively flexible in the sideways direction.

In a variant which is not shown, it is provided that there are locking means associated with the annular activation element 40 for locking the element 40 in the position in which the one or more stops are active. This locking could be with respect to the fingers 22, for example on account of the projections 42 latching in a shallow groove, or with respect to the body of the liquid container, for example with respect to the annular wall 19.

FIG. 5 shows the injection syringe 1 in the state in which it is delivered for use. The injection syringe 1 is now provided with two protective caps, namely cap 24 for the injection needle 2 and cap 25 at the other end for protecting that part of the plunger/plunger rod assembly which projects out of the liquid container. The caps 24 and 25 have to be removed before the injection syringe 1 can be used.

FIG. 6 shows the injection syringe 1 in accordance with FIG. 5 while it is sucking a liquid 9 out of a via 26. In the process, the plunger rod 7 is moved backwards in the direction indicated by the arrow in FIG. 6. If desired, the liquid 9 can be mixed or homogenized in the injection syringe 1 by the plunger rod 7 being moved to and fro a number of times.

FIG. 7 shows the injection syringe 1 in accordance with FIGS. 5 and 6 while an injection is being administered to a patient 105. As can be seen clearly from FIG. 7, the liquid container is in the process substantially completely emptied, and a coupling has been effected between the coupling means 13 at the needle mount and coupling means 15 on the plunger head.

Also, when the injection syringe 100 is completely emptied, the actuating surface 23a of the thickened portion 23 of the plunger rod 7 presses against the activation element 16 held in position, and then presses the said element 16 further inwards with respect to the annular-wall 19 of the liquid container 3. The fingers 22 are then pressed inwards and bear under prestress against the ribs 31 of the plunger rod 7.

As has already been described above, with this type of injection syringe it is provided that after injection the plunger rod 7 is retracted, with the needle 2 and mount being decoupled from the liquid container and moving into the liquid container 3. This situation is shown in FIG. 8.

It can also be seen in FIG. 8 that the plunger rod 7 has been retracted sufficiently far for the fingers 22 to move further inwards and latch into the recesses 21 in the plunger rod 7. This situation is shown in detail in FIG. 10. In this case, the stop faces 22a of the fingers 22, in this embodiment the end faces of the fingers 22, lie in the path of the stop faces 30a formed by formation 30 on the plunger rod 7. As a result, the plunger rod 7 cannot be pushed further into the liquid container 3.

As has already been indicated, in this embodiment that part of the plunger rod 7 which projects out of the liquid container can be broken off at the break-off zone 14, where the plunger rod is of locally weakened design.

FIGS. 9 and 10 show the action of the travel limiter shown in FIGS. 5-8 by means of enlarged partial views.

The right-hand side of FIG. 9 shows the position of the travel limiter when the injection syringe 1 is delivered, i.e. in the position shown in FIG. 5. The blocking finger 22 is in this case in a stress-free state.

The left-hand side of FIG. 9 shows the situation in FIG. 7, in which the thickened portion 23 of the plunger rod 7 has pushed the activation element 40 further in and is thereby pushing fingers 22 onto the plunger rod 7. The person skilled in the art will quickly understand that the liquid container 3 may comprise more than two fingers 22.

For the sake of clarity, FIG. 10 shows an enlarged partial view of the travel limiter 16 shown in FIGS. 5-8, in which the stop face 30a has moved passed the fingers 22 that block the plunger rod. FIG. 10 corresponds to the situation shown in FIG. 8.

FIG. 11 shows a perspective, cross-sectional view of the liquid container 3 as used in the injection syringe 1 in accordance with FIGS. 5-10. This fig. clearly shows the position of the fingers 22 in the stress-free state.

Finally, FIG. 12 shows a diagrammatic cross section through a further alternative injection syringe 100′ according to the invention, in which the plunger rod 7 is provided with a series of recesses 21. The recesses 21 are distributed over the length of the plunger rod 7 and alternate with knurls 27. On account of the presence of the recesses 21 and knurls 27, the plunger rod 7, after activation of the travel limiter 7, can always be moved a bit further out of the liquid container 3 (i.e. away from the opening 4), but can then no longer be moved back towards the outlet opening 4.

In other words, it is possible to provide an injection syringe with a travel limiter at the end remote from the outlet opening which is provided with a plunger rod blocker which can block the plunger rod after activation of the travel limiter. In this case, the plunger rod blocker advantageously comprises one or more inwardly directed projections. Furthermore, it is preferable for the plunger rod to comprise a recess which can interact with the plunger rod blocker.

It is preferable for the recess to be arranged at a position on the plunger rod which is such that the recess can interact with the plunger rod blocker as soon as the injection needle has been moved into the liquid container; as a result, the injection needle can no longer easily move out of the liquid container.

The plunger rod particularly preferably has a series of recesses distributed over the longitudinal axis of the plunger rod. The recesses are advantageously in this case designed in such a manner that—after activation of the travel limiter—the plunger rod can still be moved a small amount further away from the outlet opening of the liquid container, but then can no longer be moved back outwards (i.e. via the outlet opening); the recesses on the plunger rod in this case alternate with a series of small knurls.

According to a further preferred embodiment, the injection syringe according to the invention has a spring member which can move the needle mount with injection needle into the liquid container after the blocking member has been unblocked (by pulling or pushing, depending on the position of the spring member in the injection syringe). The person skilled in the art will readily understand that the spring member may be a spring but also be another form of resilient member, such as for example a rubber band or the like.

In particular, the present invention relates to a travel limiter which is in the form of a substantially cylindrical member, which member, in the vicinity of the end which faces the outlet opening in use, comprises one or more outwardly directed claws, and in the vicinity of the end remote from the outlet opening is provided with one or more inwardly facing projections.

1. Injection syringe with retractable injection needle, comprising:
a liquid container having a needle opening;
a plunger/plunger rod assembly, which is moveable over a certain travel in the liquid container (3) and comprises a plunger rod, plunger and a plunger head;
an injection needle with needle mount which, in an active position, projects out of the liquid container through the needle opening and, in a retracted position, is located fully within the liquid container;
a travel limiter which comes into action after an injection has been administered using the injection syringe and subsequently the injection needle with needle mount has been moved into the retracted position by the plunger/plunger rod assembly being retracted, after which it restricts the plunger/plunger rod assembly in the event of a movement towards the needle opening, in such a manner that the injection needle cannot be pushed out of the liquid container,
wherein the travel limiter comprises a stop mechanism having a stop face associated with the plunger/plunger rod assembly, and having a stop face associated with the liquid container, it being possible for at least one stop face to be displaced between an initial, inactive position, in which an injection can be administered using the injection syringe, and an active position, in which the stop faces actively come into contact with one another and thereby limit the travel of the plunger/plunger rod assembly.

2. Injection syringe according to claim 1, in which a stop face associated with the liquid container is formed by at least one moveable stop which is fitted to the liquid container.

3. Injection syringe according to claim 2, in which a moveable stop is a resilient stop.

4. Injection syringe according to claim 2, in which a stop is integral with the liquid container.

5. Injection syringe according to claim 2, in which a stop is arranged on that side of the liquid container which is remote from the needle opening.

6. Injection syringe according to one claim 2, in which a plurality of stops, which are moveable between an outer, inactive position and an inner, active position, are arranged around the opening meant for the plunger/plunger rod assembly on that side of the liquid container which is remote from the needle opening.

7. Injection syringe according to claim 5, in which an activation element which actuates the one or more moveable stops is arranged on that side of the liquid container which is remote from the needle opening.

8. Injection syringe according to claim 7, in which the activation element is designed to come into contact with an associated actuation surface of the plunger/plunger rod assembly, preferably when the plunger/plunger rod assembly is displaced fully inward in order to deliver an injection.

9. Injection syringe according to claim 7, in which the activation element is an element which is moveable with respect to the liquid container and is moveable as a result of an actuating surface of the plunger/plunger rod assembly coming into contact with it.

10. Injection syringe according to claim 9, in which the activation element has associated locking means for locking the activation element in the position in which the one or more stops are active.

11. Injection syringe according to claim 6, in which the liquid container, at the end remote from the needle opening, has an annular wall, inside which the activation element is arranged.

12. Injection syringe according to claim 1, in which a stop face associated with the liquid container is formed by a stop element that is slideably guided on the plunger rod, the stop element and the liquid container being provided with interacting coupling means which are designed in such a manner that when the plunger/plunger rod assembly moves inwards in order to discharge liquid from the injection syringe, the stop element is coupled to the liquid container.

13. Injection syringe according to claim 12, in which the stop face associated with the plunger/plunger rod assembly, during the retraction of the plunger/plunger rod assembly, moves past the one or more stop faces of the stop element, in such a manner that in the event of the assembly being moved inwards again the stop faces come into contact with one another.

14. Injection syringe according to claim 1, in which a stop face of the plunger/plunger rod assembly is arranged at a recess, which is open towards the side, in the assembly.

15. Injection syringe according to claim 14, in which part of the plunger rod is cross-shaped in cross section, with longitudinal ribs and longitudinal grooves, a stop face of the assembly being formed by a stop formation in a longitudinal groove.

16. Injection syringe according to claim 13, in which the coupling means comprise one or more recesses in the liquid container and one or more hook members on the stop element.

17. Injection syringe according to claim 1, in which the plunger rod is provided with a locally weakened section as a break-off zone.

18. Injection syringe according to claim 1, in which coupling means are provided for coupling the plunger/plunger rod assembly to the needle mount after the liquid container has been substantially emptied.

19. Plunger rod described as a component of the injection syringe according to claim 1.

20. Travel limiter described as a component of the injection syringe according to claim 1.

21. Liquid container described as a component of the injection syringe according to claim 1.

22. Set composed of a liquid container, a plunger rod (7) and a travel limiter described as one or more components in claim 1.
